(19) United States
(12) Patent Application Publication
(10) Pub. No.: US 2023/0384331 A1
(43) Pub. Date: Nov. 30, 2023

(54) BLOOD CHARACTERISTICS EVALUATION APPARATUS, BLOOD CHARACTERISTICS EVALUATION METHOD, AND NON-TRANSITORY COMPUTER READABLE RECORDING MEDIUM

(71) Applicant: CANON MEDICAL SYSTEMS CORPORATION, Tochigi (JP)

(72) Inventors: Kohei WATANABE, Utsunomiya (JP); Tomoki FUJITO, Nasushiobara (JP)

(73) Assignee: CANON MEDICAL SYSTEMS CORPORATION, Tochigi (JP)

(21) Appl. No.: 18/446,588

(22) Filed: Aug. 9, 2023

Related U.S. Application Data
(63) Continuation of application No. PCT/JP2022/003384, filed on Jan. 28, 2022.

(30) Foreign Application Priority Data
Feb. 9, 2021 (JP) 2021-019068

(51) Int. Cl. G01N 33/86; G01N 21/55; G01N 21/53

(52) U.S. Cl. CPC G01N 33/86; G01N 21/55; G01N 21/53; G01N 2400/40; G01N 2021/558

(57) ABSTRACT

A blood characteristics evaluation apparatus (1) according to one embodiments includes processing circuitry (12) configured to cause a foaming phenomenon to occur in the blood sample, measure a foaming state in the blood sample, and calculate an index indicating a coagulation tendency of the blood sample based on a measurement result of the foaming state in the blood sample.

### CROSS-REFERENCE TO RELATED APPLICATIONS

This application is a continuation of PCT international application Ser. No. PCT/JP2022/003384 filed on Jan. 28, 2022 which designates the United States, incorporated herein by reference, and which claims the benefit of priority from Japanese Patent Application No. 2021-019068 filed on Feb. 9, 2021, the entire contents of which are incorporated herein by reference.

### FIELD

Embodiments described herein relate generally to a blood characteristics evaluation apparatus, a blood characteristics evaluation method, and a non-transitory computer readable recording medium.

### BACKGROUND

A technology for evaluating blood characteristics of a patient and using the evaluation for making a treatment plan, developing a procedure, or the like is known. For example, in some patients, abnormal coagulation or fibrinolysis may occur in a procedure using an artificial heart-lung machine or an acute thrombosis may occur in Drug-Eluting Stent (DES) placement; however, by evaluating the blood characteristics, it becomes possible to consider countermeasures in advance.

Patent Literature 1: Japanese Laid-open Patent Publication No. 2011-154036

One of problems to be solved by embodiments disclosed in the present specification and drawing is to provide a new method for evaluating blood characteristics. However, problems to be solved by the embodiments disclosed in the present specification and the drawings are not limited to the problem as described above. Problems corresponding to effects achieved by components illustrated in the embodiments described below may be regarded as other problems.

### BRIEF DESCRIPTION OF THE DRAWINGS

FIG. 1 is a diagram illustrating a configuration example of a blood characteristics evaluation apparatus according to a first embodiment;

FIG. 2 is a diagram illustrating a configuration example of flow paths according to the first embodiment;

FIG. 3 is a diagram illustrating an overview of a process performed by processing circuitry according to the first embodiment;

FIG. 4 is a diagram illustrating an example of evaluation of characteristics of blood samples according to the first embodiment;

FIG. 5 is a flowchart illustrating an example of a process performed by processing circuitry of the blood characteristics evaluation apparatus according to the first embodiment; and

FIG. 6 is a diagram illustrating a configuration example of flow paths according to a second embodiment.

### DETAILED DESCRIPTION

A blood characteristics evaluation apparatus according to one embodiments includes processing circuitry configured to cause a foaming phenomenon to occur in the blood sample, measure a foaming state in the blood sample, and calculate an index indicating a coagulation tendency of the blood sample based on a measurement result of the foaming state in the blood sample.

Embodiments of the blood characteristics evaluation apparatus, a blood characteristics evaluation method, and non-transitory computer readable recording medium will be described in detail below with reference to the accompanying drawings.

### First Embodiment

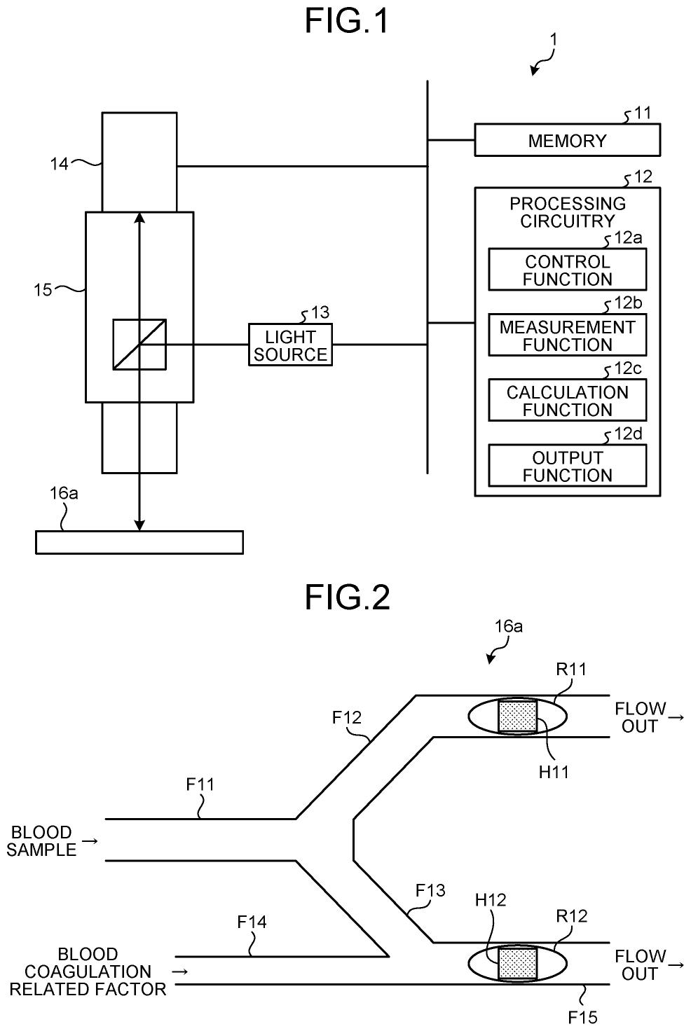

FIG. 1 is a diagram illustrating a configuration example of a blood characteristics evaluation apparatus 1 according to a first embodiment. For example, the blood characteristics evaluation apparatus 1 includes a memory 11, processing circuitry 12, a light source 13, a camera 14, a microscope 15, and a sample holder 16a.

The memory 11 is implemented by, for example, a semiconductor memory device, such as a Random Access Memory (RAM) or a flash memory, a hard disk, an optical disk, or the like. For example, the memory 11 stores therein a program that causes circuitry included in the blood characteristics evaluation apparatus 1 to implement functions.

The processing circuitry 12 executes a control function 12a, a measurement function 12b, a calculation function 12c, and an output function 12d, and controls entire operation of the blood characteristics evaluation apparatus 1. The control function 12a is one example of a control unit. The measurement function 12b is one example of a measurement unit. The calculation function 12c is one example of a calculation unit. The output function 12d is one example of an output unit.

For example, the processing circuitry 12 reads a program corresponding to the control function 12a from the memory 11, executes the program, and controls flow of a blood sample in flow paths that are arranged in the sample holder 16a. Further, the control function 12a causes a foaming phenomenon to occur in the blood sample. Furthermore, the processing circuitry 12 reads a program corresponding to the measurement function 12b from the memory 11, executes the program, controls operation of the light source 13, the camera 14, and the microscope 15, and measures a foaming state in the blood sample. Moreover, the processing circuitry 12 reads a program corresponding to the calculation function 12c, executes the program, and evaluates characteristics of the blood sample based on a measurement result of the foaming state. Furthermore, the processing circuitry 12 reads a program corresponding to the output function 12d, executes the program, and outputs an evaluation result obtained by the calculation function 12c. Details of processes performed by the control function 12a, the measurement function 12b, the calculation function 12c, and the output function 12d will be described later.

The light source 13, the camera 14, and the microscope 15 are examples of a measurement apparatus that measures a foaming state in the blood sample. Meanwhile, FIG. 1 is just one example, and a method of measuring the foaming state in the blood sample may be modified in various different modes.

The light source 13 is a light emitting apparatus that assists image capturing performed by the camera 14. For example, the light source 13 is an Electronic Flash apparatus that emits light at the time of image capturing performed by the camera 14 under the control of the processing circuitry 12.

The camera 14 captures an image of the blood sample in the sample holder 16a. Specifically, the camera 14 captures an image of a region in which the foaming phenomenon has occurred in the blood sample under the control of the processing circuitry 12. Meanwhile, it may be possible to continuously capture images of a plurality of frames with respect to the region in which the foaming phenomenon has occurred. In other words, the camera 14 may capture a moving image. Furthermore, the blood characteristics evaluation apparatus 1 may include, as the camera 14, a high speed camera.

For example, by causing the light source 13 to emit light in synchronization with a foaming timing, it is possible to cause the camera 14 to capture an image of foaming. In this case, it may be possible to add a delay time (delay) to a light emitting timing of the light source 13 in order to capture the image at an arbitrary timing in the process from foaming to defoaming. Further, it may be possible to cause the light source 13 to continuously emit light in accordance with the foaming timing, and cause the camera 14 to continuously capture images of a plurality of foaming states.

The microscope 15 optically enlarges the region in which the foaming phenomenon has occurred in the blood sample. The camera 14 is able to capture an enlarged image of the region in which the foaming phenomenon has occurred by performing image capturing via the microscope 15.

In the blood characteristics evaluation apparatus 1 illustrated in FIG. 1, each of processing functions is stored in the memory 11 in a computer executable mode. The processing circuitry 12 is a processor that reads programs from the memory 11, executes the programs, and implements a function corresponding to each of the programs. In other words, the processing circuitry 12 that has read a program has a function corresponding to the read program.

Meanwhile, it is explained in FIG. 1 that the single processing circuitry 12 implements the control function 12a, the measurement function 12b, the calculation function 12c, and the output function 12d, but it may be possible to construct the processing circuitry 12 by combination of a plurality of independent processors and cause each of the processors to execute a program and implement a function. Further, each of the processing functions included in the processing circuitry 12 may be implemented by being appropriately distributed or integrated into one or more processing circuitry. Furthermore, the processing circuitry 12 may implement the functions by using a processor of an external apparatus that is connected via a network. For example, the processing circuitry 12 implements each of the functions illustrated in FIG. 1 by reading a program corresponding to each of the functions from the memory 11, executing the program, and using, as a calculation resource, a server group (cloud) that is connected to the blood characteristics evaluation apparatus 1 via a network.

Thus, the configuration example of the blood characteristics evaluation apparatus 1 according to the present embodiment has been described above. With the configuration as described above, the blood characteristics evaluation apparatus 1 provides a new method for evaluating blood characteristics.

Specifically, the control function 12a causes a foaming phenomenon to occur in the blood sample. Further, the measurement function 12b measures a foaming state in the blood sample. Furthermore, the calculation function 12c evaluates characteristics of the blood sample based on the measurement result. Here, the foaming phenomenon is sensitive to a difference in characteristics, such as viscoelasticity, surface tension, or composition, of the blood sample. Therefore, the processing circuitry 12 is able to evaluate the characteristics of the blood sample with high accuracy by measuring the foaming state. Moreover, the processing circuitry 12 is able to simply and easily evaluate the characteristics of the blood sample without a need of cumbersome processes.

Control on the blood sample by the control function 12a will be described below with reference to FIG. 2. FIG. 2 is a diagram illustrating a configuration example of flow paths according to the first embodiment. The control function 12a causes the blood sample to flow in the flow paths illustrated in FIG. 2, and causes a foaming phenomenon to occur in a part of the blood sample.

As illustrated in FIG. 2, the sample holder 16a includes flow paths that allow the blood sample to flow. Specifically, the sample holder 16a includes, as the flow paths that allow the blood sample to flow, a groove that is arranged on a surface, a tunnel-shaped internal space, a tube, or the like. For example, in the sample holder 16a, the flow paths are arranged by a certain method, such as Photolithography, etching, laser processing, machining including cutting, casting, or 3D printing. A material of the sample holder 16a is not specifically limited, but a material with high transmittance is preferable at least in the vicinity of a part in which the foaming phenomenon is caused to occur in the blood sample. For example, the sample holder 16a may be made by using glass or acrylic resin, such as Polymethyl Methacrylate (PMMA), in the vicinity of the part in which the foaming phenomenon is caused to occur in the blood sample. With use of the material with high transmittance, it is possible to optically measure the foaming phenomenon as will be described later.

For example, the control function 12a causes the blood sample to flow in a flow path F11 illustrated in FIG. 2. Meanwhile, the blood sample may be untreated blood that is drawn from a patient or may be treated blood. The treated blood is, for example, blood (blood plasm) from which blood cells are removed, or the like.

As one example, the blood sample is stored in a syringe (not illustrated) that includes a barrel and a plunger. In this case, the control function 12a is able to control a flow volume and a flow rate of the blood sample that flows in the flow path F11 by controlling operation of the plunger with respect to the barrel.

Meanwhile, the control function 12a may perform pretreatment on the blood sample before the blood sample is caused to flow in the flow path F11. For example, the control function 12a adjusts the number of blood cells in the blood sample to a predetermined value before the blood sample flows in the flow path F11. As one example, the control function 12a counts the number of blood cells in the blood sample by an arbitrary blood cell counter, and dilutes the blood sample by adding a diluent such that the number of blood cells per unit volume reaches a predetermined value. The diluent is not specifically limited, and may be saline, phosphate buffer solution, citrate buffer solution, or aqueous solution containing various additives, such as protein, saccharides, calcium, magnesium, or other metal ions, or non-aqueous solution. Further, the blood cell counter may be configured to be connected before the blood sample flows in the flow path F11. Furthermore, for example, the control function 12a may stir the blood sample before the blood sample flows in the flow path F11. In other words, the control function 12a performs stirring before measurement in order to maintain equal dispersion of blood cells in the blood sample. Through the pretreatment as described above, it is possible to unify measurement conditions and improve accuracy of blood evaluation.

Moreover, the control function 12a may perform pretreatment to prevent coagulation in the flow paths. For example, the control function 12a adds a factor for preventing coagulation to the blood sample at an arbitrary time point, such as at the time of blood drawing, before flow-in of the blood sample is started. The control function 12a may use, as the factor for preventing coagulation, an anticoagulant agent, such as sodium citrate, Ethylene-Diamine-Tetraacetic Acid (EDTA), warfarin, heparin, low-molecular weight heparin, a factor IIa inhibitor, or a factor Xa inhibitor, for example. As another example, the control function 12a may use, as the factor for preventing coagulation, an arbitrary factor that prevents blood coagulation or reduces blood coagulation. Furthermore, if sodium citrate or EDTA is used in advance as the factor for preventing coagulation, it may be possible to add calcium before measurement in order to recover coagulation property.

In the example illustrated in FIG. 2, the flow path F11 is branched into a flow path F12 and a flow path F13. In other words, the blood sample that flows in the flow path F11 is divided for the flow path F12 and the flow path F13. The flow path F12 is one example of a first flow path. The flow path F13 is one example of a second flow path.

The control function 12a causes the foaming phenomenon to occur in the blood sample in the flow paths. For example, the control function 12a applies thermal energy to the blood sample from a heating part that is included in the sample holder 16a and causes the foaming phenomenon to occur. For example, the sample holder 16a includes, as the heating part, a heater H11 at a position corresponding to a region R11 in FIG. 2. As one example, the heater H11 is a thermoelectric conversion element, such as cobalt silicide (CoSi2). In this case, the control function 12a controls power supply to the thermoelectric conversion element to boil a part of the blood sample, so that it is possible to cause the foaming phenomenon to occur. For example, the control function 12a is able to cause the foaming phenomenon to occur in the blood sample that flows in the region R11 in the flow path F12 by applying thermal energy to the blood sample from the heater H11.

More specifically, if heating is performed by the thermoelectric conversion element, film boiling occurs at an interface. Specifically, at the moment at which the blood sample reaches heating limit temperature, a plurality of bubbles are generated at an interface between the thermoelectric conversion element and the blood sample and rapidly grow while coalescing. At this time, pressure in the bubbles act as impulse and especially high pressure is generated at the moment of foaming. The bubbles start to grow due to the force as described above, and continue to grow due to inertia of the blood sample even after the bubble pressure disappears, so that a negative pressure state is already generated at the time of maximum foaming. Therefore, a time needed from foaming to defoaming is reduced, and an influence of the foaming phenomenon on the blood sample is reduced. In other words, the control function 12a causes the foaming phenomenon to occur by film boiling, so that it is possible to further improve accuracy of blood evaluation.

Meanwhile, it may be possible to cause the foaming phenomenon to occur by a method other than the method of applying the thermal energy. For example, it may be possible to arrange a piezoelectric element at a position corresponding to the region R11 in FIG. 2, generate ultrasound waves, and cause the foaming phenomenon to occur by pressure fluctuation due to the ultrasound waves. In other words, the control function 12a is able to use an arbitrary foam-inducing element, such as a thermoelectric conversion element or a piezoelectric element, and cause the foaming phenomenon to occur at the position corresponding to the region R11.

The measurement function 12b measures the foaming state in the blood sample. For example, the measurement function 12b controls operation of the light source 13, the camera 14, and the microscope 15, and captures an image of the region R11 in which the foaming phenomenon has occurred. Specifically, the measurement function 12b activates the light source 13 and causes the camera 14 to capture an enlarged image of the region R11 via the microscope 15 while causing the light source 13 to emit light. Further, the measurement function 12b measures the foaming state based on the captured image of the region R11.

As one example, the control function 12a applies pulse voltage to the thermoelectric conversion element and causes the foaming phenomenon to occur in the blood sample. Further, after the control function 12a has applied the pulse voltage, the measurement function 12b captures the image of the region R11 after a lapse of an arbitrary delay time (delay). The delay time is adjusted in accordance with a standard time needed until a bubble generated in the blood sample has a maximum size after application of the pulse voltage to the thermoelectric conversion element, for example.

Alternatively, after the control function 12a has applied the pulse voltage, the measurement function 12b may cause the camera 14 to continuously capture images of the region R11 for a predetermined period of time. For example, the measurement function 12b causes the light source 13 to continuously emit light in accordance with the foaming timing, and causes the camera 14 to continuously capture images of a plurality of foaming states. As one example, the measurement function 12b continuously capture a plurality of images at a predetermined frame rate during a standard period of time until defoaming after bubbles are generated in the blood sample due to the application of the pulse voltage. Meanwhile, the control function 12a may repeat foaming and deforming by continuously applying the pulse voltage a plurality of number of times. Further, the measurement function 12b may repeatedly capture the foaming state at a predetermined timing since application of the pulse voltage. In other words, the control function 12a periodically causes foaming and defoaming to occur, and the measurement function 12b causes the light source 13 to emit light and causes the camera 14 to perform image capturing in synchronization with the period, so that it is possible to repeatedly capture images of the foaming state in the same phase. In other words, the measurement function 12b is able to perform stop-motion-capture by electronic flash photography.

Furthermore, the measurement function 12b measures a foaming state based on the image of the region R1l. For example, the measurement function 12b measures a size of a bubble that is generated in the region R11 as the foaming state. Meanwhile, the bubble size is not specifically limited as long as information indicates the size of the bubble. For example, the bubble size may be a diameter, a circumferential length, an area, a volume, the number of pixels, or the like of the bubble.

Meanwhile, the bubble in the image is not always a circle, but may be deformed from a circle. In this case, the measurement function 12b may identify a long axis or a short axis of the bubble, and adopts a long diameter or a short diameter as the bubble size, for example. Furthermore, if the plurality of images of the region R11 are captured, the measurement function 12b may identify an image in which the bubble size is the largest and adopt the bubble size in the identified image as a measurement result of the foaming state, for example. Alternatively, the measurement function 12b may adopt a statistical value, such as an average value, of the bubble sizes that are measured based on the plurality of images, as the measurement result of the foaming state.

Furthermore, the measurement function 12b may eliminate an outlier from the measurement result of the foaming state. For example, the measurement function 12b measures the bubble size of each of bubbles that are generated by repeating heating a plurality of number of times or bubble sizes of some of the bubbles. Moreover, the thermoelectric conversion elements may be arranged in series or in parallel in different flow paths, apply pulse voltage to the plurality of thermoelectric conversion elements at the same timing or different timings to generate a plurality of bubbles, and measure bubble sizes of the respective bubbles. Furthermore, the measurement function 12b eliminates an outlier from measurement values of the plurality of bubbles. As one example, the measurement function 12b sets a predetermined standard deviation as a threshold, identifies an outlier by comparison with the standard deviation, and eliminates the outlier. In this case, the calculation function 12c evaluates the characteristics of the blood sample based on the measurement result from which the outlier is eliminated, so that it is possible to further improve evaluation accuracy.

Moreover, as illustrated in FIG. 2, the control function 12a causes a blood coagulation related factor to flow in a flow path F14. The blood coagulation related factor is a component or a substance related to blood coagulation. As one example, the blood coagulation related factor may be anticoagulant or a coagulation inhibitor, such as heparin, EDTA, sodium citrate, coumarin derivative (for example, warfarin or dicoumarol), a tissue factor pathway inhibitor (TFPI), antithrombin III, lupus anticoagulant, nematode anticoagulant protein c2 (NAPc2), VIIa factor in which active site is blocked (VIIai factor), an IXa factor inhibitor, an Xa factor inhibitor (including fondaparinux, idraparinux, DX-9065a, and razaxaban (DPC906)), a Va factor and VIIIa factor inhibitor (including activated protein C (APC) and soluble thrombomodulin), a thrombin inhibitor (including hirudin, bivalirudin, argatroban, and ximelagatran), a C1 esterase inhibitor, or a heparin cofactor 2; for example, a fibrinolytic factor, such as plasminogen, t-PA, pro-urokinase, PAI-1, or u-PA, and a fibrinolysis inhibitor. Heparin will be described below as one example of the blood coagulation related factor. For example, the control function 12a causes, s the blood coagulation related factor, a heparin solution containing heparin at a predetermined concentration to flow in the flow path F14.

As illustrated in FIG. 2, the flow path F13 and the flow path F14 are merged into a flow path F15. Specifically, in the flow path F15, the blood sample that flows in the flow path F13 and the heparin solution that flows in the flow path F15 flow in a mixed manner. In the following, the blood sample in which the blood coagulation related factor, such as the heparin solution, is mixed may be described as a composite sample. The flow path F14 is one example of a third flow path. Further, the flow path F15 is one example of a fourth flow path. Meanwhile, it may be possible to arrange a component that can fully mix the blood sample and the heparin solution in a portion in which the flow paths F13 and F14 are merged on the upstream side of a region R12. Although not specifically limited, for example, it may be possible to set a flow path length that is enough for mixing, or it may be possible to arrange a meander flow path to promote mixing.

Similarly to the case of the region R1l, the control function 12a causes a foaming phenomenon to occur in the region R12 in the composite sample that flows in the flow path F15. For example, the sample holder 16a includes, as a heating part, a heater H12 at a position corresponding to the region R12 in FIG. 2. Further, the control function 12a applies thermal energy to the composite sample from the heater H12, and causes the foaming phenomenon to occur in the composite sample that flows in the region R12 in the flow path F15. Furthermore, the measurement function 12b measures a foaming state in the composite sample. For example, the measurement function 12b captures an image of the region R12 in which the foaming phenomenon has occurred, and measures a bubble size based on the image. Meanwhile, only a single measurement apparatus that includes the light source 13, the camera 14, and the microscope 15 is illustrated in FIG. 1, but the blood characteristics evaluation apparatus 1 may include the measurement apparatus for each of the region R11 and the region R12.

As one example, the measurement function 12b measures the foaming state in the blood sample that flows in the flow path F12 and the foaming state in the composite sample that flows in the flow path F15 at approximately the same time. Specifically, the control function 12a applies thermal energy to the region R11 and the region R12 at approximately the same time and causes the foaming phenomena to occur. Further, the measurement function 12b capture the image of the region R11 and the image of the region R12 at approximately the same time. Furthermore, the measurement function 12b is able to measure the bubble sizes in the blood sample and the composite sample at approximately the same time based on the images.

Here, the measurement function 12b may repeatedly measure the foaming state in the composite sample while changing the blood coagulation related factor. For example, after causing a solution containing heparin at a predetermined concentration to flow in the flow path F14 and measuring the foaming state, the control function 12a causes a solution containing heparin at a different concentration or a solution containing a different kind of anticoagulant factor to flow in the flow path F14, and the measurement function 12b measures the foaming state in the composite sample again.

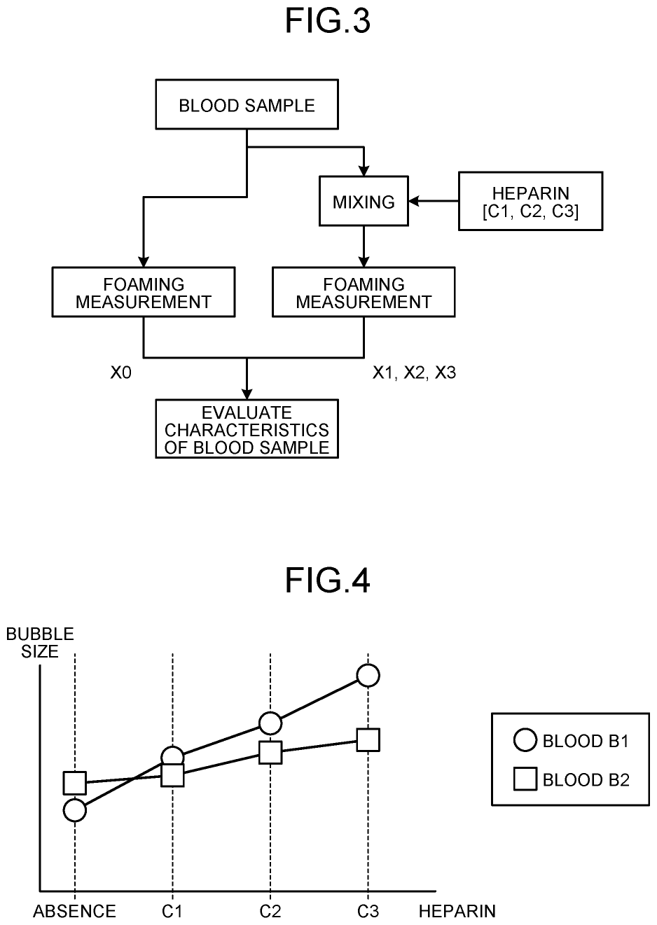

For example, the measurement function 12b acquires a measurement result X0 of the foaming state in the blood sample as illustrated in FIG. 3. The measurement result X0 is, for example, a bubble size that is measured with respect to the blood sample that flows in the flow path F12. Meanwhile, FIG. 3 is a diagram illustrating an overview of the process performed by the processing circuitry 12 according to the first embodiment.

Further, the measurement function 12b acquires a measurement result of the foaming state of the composite sample in which the blood sample and the heparin are mixed. For example, the measurement function 12b acquires a measurement result X1 of the foaming state of the composite sample in which the blood sample and heparin solution L11 containing the heparin are mixed. The concentration or a flow volume of the heparin in the heparin solution L11 is adjusted such that the concentration of the heparin in the composite sample as a mixture of the blood sample and the heparin solution L11 reaches a concentration C1. Subsequently, the measurement function 12b acquires a measurement result X2 of the foaming state of the composite sample in which the blood sample and a heparin solution L12 containing heparin are mixed. A concentration or a flow volume of the heparin in the heparin solution L12 is adjusted such that the concentration of the heparin in the composite sample as a mixture of the blood sample and the heparin solution L12 reaches a concentration C2. Then, the measurement function 12b acquires a measurement result X3 of the foaming state of the composite sample in which the blood sample and a heparin solution L13 containing heparin are mixed. A concentration or a flow volume of the heparin in the heparin solution L13 is adjusted such that the concentration of the heparin in the composite sample as a mixture of the blood sample and the heparin solution L13 reaches a concentration C3. Meanwhile, the concentration C2 is higher than the concentration C1, and the concentration C3 is higher than the concentration C2. Further, the calculation function 12c evaluates the characteristics of the blood sample based on the measurement results X0 to X3.

A process of evaluating the characteristics of the blood sample by the calculation function 12c will be described below with reference to FIG. 4. FIG. 4 is a diagram illustrating an example of evaluation of characteristics of blood samples according to the first embodiment. FIG. 4 illustrates, as examples of the blood samples, two examples such as blood B1 that is collected from a patient P1 and blood B2 that is collected from a patient P2. Further, a horizontal axis in FIG. 4 represents the concentration of the heparin. Furthermore, a vertical axis in FIG. 4 represents the measured bubble size.

In the horizontal axis in FIG. 4, “absence” of heparin indicates the measurement result X0. Specifically, “absence” of heparin indicates a bubble size that is measured with respect to the blood sample that flows in the flow path F12. Further, heparin “C1” indicates the measurement result X1 that is obtained by measuring the foaming state of the composite sample in which the heparin solution L11 and the blood sample are mixed such that the concentration of the heparin reaches the concentration C1. Furthermore, heparin “C2” indicates the measurement result X2 that is obtained by measuring the foaming state of the composite sample in which the heparin solution L12 and the blood sample are mixed such that the concentration of the heparin reaches the concentration C2. Moreover, heparin “C3” indicates the measurement result X3 that is obtained by measuring the foaming state of the composite sample in which the heparin solution L13 and the blood sample are mixed such that the concentration of the heparin reaches the concentration C3. Meanwhile, if heparin has been added to the blood sample as a pre-treatment to prevent coagulation in the flow path, it may be possible to perform plotting in FIG. 4 while taking into account the amount of the heparin that has been added in the pre-treatment.

As illustrated in FIG. 4, the blood B1 and the blood B2 have different responsiveness with respect to the heparin. Specifically, in the blood B1, the bubble size highly tends to increase with an increase in the concentration of the heparin. Here, the bubble size decreases with an increase in viscoelasticity of liquid; however, viscoelasticity of the blood B1 highly tends to decrease with an increase in the concentration of the heparin, so that the blood B1 is regarded as blood with a high coagulation tendency. In contrast, the blood B2 is regarded as blood with a low coagulation tendency because variation in the bubble size with respect to the concentration of the heparin is small.

The calculation function 12c is able to evaluate the characteristics of each of the blood samples based on the responsiveness with respect to the heparin as described above. For example, the calculation function 12c obtains a regression equation with respect to the plot (scatter diagram) of the blood B1 as illustrated in FIG. 4, and calculates a slope of the regression equation. Similarly, the calculation function 12c calculates a slope of the regression equation with respect to the plot of the blood B2. The slopes of the regression equations as described above are one example of an index that indicates a coagulation tendency of the blood sample.

Here, the calculation function 12c may evaluate the characteristics of the blood sample while taking into account the number of blood cells in each of the blood samples. For example, the calculation function 12c calculates an index that indicates the coagulation tendency of the blood sample based on the measurement result of the foaming state and the number of blood cells in the blood sample.

For example, the calculation function 12c first calculates the slope of the regression equation between the concentration of the heparin and the bubble size. Further, the calculation function 12c calculates a value that is obtained by correcting the calculated slope in accordance with the number of blood cells, as the index indicating the coagulation tendency of the blood sample. For example, in general, it is known that blood has higher viscosity with an increase in the number of blood cells. Therefore, if the responsiveness with respect to the heparin is approximately the same, the possibility that a blood flow is disrupted increases with an increase in the number of blood cells and coagulation is likely to occur. Therefore, the calculation function 12c calculates a value that is obtained by correcting the slope calculated from the regression equation such that the slope increases with an increase in the number of blood cells, as the index indicating the coagulation tendency of the blood sample.

The output function 12d outputs an evaluation result obtained by the calculation function 12c. For example, the output function 12d displays the evaluation result on a display apparatus that is connected to the blood characteristics evaluation apparatus 1. Meanwhile, the display apparatus is, for example, a display, such as a liquid crystal display, a Cathode Ray Tube (CRT) display, or a touch panel. For example, the output function 12d displays a value, a graph, or the like that indicates the slope of the regression equation calculated by the calculation function 12c.

Meanwhile, the output function 12d may display an error range in addition to displaying the evaluation result. For example, the output function 12d displays the slope of the regression equation between the concentration of the heparin and the bubble size as the evaluation result obtained by the calculation function 12c. Further, the output function 12d displays the error range with respect to the slope. In this case, the error range may be calculated in accordance with magnitude of an error that occurs when the plurality of plots illustrated in FIG. 4 are approximated by the regression equation, for example. The output function 12d may illustrate the error range by an error bar or the like or may display the error range by text.

Further, the output function 12d may output the evaluation result obtained by the calculation function 12c in various forms other than the display. Specifically, the output function 12d provides the evaluation result obtained by the calculation function 12c to a user, such as a doctor, in a direct or indirect manner. For example, the output function 12d may control a projector and projects the evaluation result obtained by the calculation function 12c on an arbitrary plane. Furthermore, the output function 12d may print out the evaluation result. Moreover, the output function 12d may give a notice of the evaluation result to the user by voice or the like. Furthermore, the output function 12d may transmit and store the evaluation result to and in an external server. As one example, the output function 12d registers the evaluation result on a system, such as a Hospital Information System (HIS). In this case, the user is able to arbitrarily access the system and refer to the evaluation result.

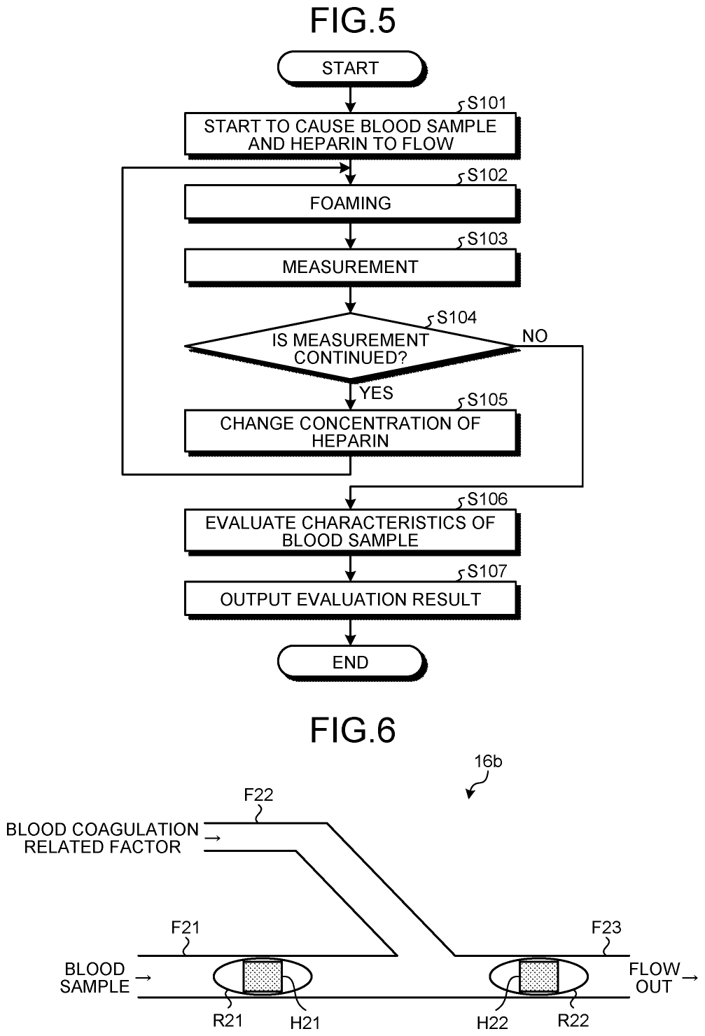

An overview of a series of processes performed by the blood characteristics evaluation apparatus 1 will be described below with reference to FIG. 5. FIG. 5 is a flowchart illustrating an example of the process performed by the processing circuitry 12 of the blood characteristics evaluation apparatus 1 according to the first embodiment. Step S101, Step S102, Step S104, and Step S105 correspond to the control function 12a. Step S103 corresponds to the measurement function 12b. Step S106 corresponds to the calculation function 12c. Step S107 corresponds to the output function 12d.

First, the processing circuitry 12 starts to cause the blood sample and the heparin to flow in the flow paths of the sample holder 16a (Step S101). For example, the processing circuitry 12 starts to cause the blood sample to flow in the flow path F11 and cause the heparin to flow in the flow path F14 at approximately the same time.

Subsequently, the processing circuitry 12 causes the foaming phenomenon to occur in each of the samples (Step S102). For example, the processing circuitry 12 applies thermal energy to the blood sample that flows in the flow path F12, and causes the foaming phenomenon to occur. Further, the processing circuitry 12 applies thermal energy to the composite sample that flows in the flow path F15, and causes the foaming phenomenon to occur.

Then, the processing circuitry 12 measures foaming states (Step S103). For example, the processing circuitry 12 controls the light source 13, the camera 14, and the microscope 15 to capture images of the regions in which the foaming phenomena have occurred. For example, the processing circuitry 12 captures images of the region R11 and the region R12 illustrated in FIG. 2. Further, the processing circuitry 12 measures bubble sizes based on the captured images.

Subsequently, the processing circuitry 12 determines whether to continue the measurement (Step S104). If the measurement is continued (Yes at Step S104), the processing circuitry 12 changes the concentration of the heparin (Step S105), and the process goes to Step S102 again. For example, after causing the heparin solution L11 to flow in the flow path F14 and performing the measurement, the processing circuitry 12 changes the sample that flow in the flow path F14 to the heparin solution L12 and goes to Step S102 again.

In contrast, if the measurement is not continued (No at Step S104), the processing circuitry 12 evaluates the characteristics of the blood sample based on the measurement result of the foaming state (Step S106). For example, the processing circuitry 12 obtains a regression equation between the concentration of the heparin and the bubble size, and calculates the slope of regression equation as the index indicating the coagulation tendency of the blood sample. Then, the processing circuitry 12 outputs the evaluation result of the blood sample and terminates the process (Step S107).

As described above, according to the first embodiment, the control function 12a causes the foaming phenomenon to occur in the blood sample. Further, the control function 12a causes the foaming phenomenon to occur in the composite sample in which the blood sample and the blood coagulation related factor are mixed. Furthermore, the measurement function 12b measures the foaming state in the blood sample and the foaming state in the composite sample. Moreover, the calculation function 12c evaluates the characteristics of the blood sample based on measurement results of the foaming state in the blood sample and the foaming state in the composite sample. In other words, the blood characteristics evaluation apparatus 1 according to the first embodiment provides a new method for evaluating the blood characteristics.

Here, the foaming phenomenon is sensitive to a difference in the characteristics of the blood sample, but the blood characteristics evaluation apparatus 1 is able to evaluate the characteristics of the blood sample with high accuracy based on the measurement result of the foaming state. Further, as illustrated in FIG. 5, a process that takes a long time is not needed before evaluation of the characteristics of the blood sample, and a special measurement apparatus or the like that needs a high manufacturing cost is not needed. In other words, the blood characteristics evaluation apparatus 1 is able to simply and easily evaluate the characteristics of the blood sample.

As another method for evaluating blood, a method of catching fluctuation of electrical resistance of a piezoresistive element. Specifically, it is possible to detect, as electrical resistance, deflection of a flexible element, such as a piezoresistive element, and evaluate viscoelasticity or the like of the blood sample. However, a physical mutation amount as a measurement target in this method is minute, so that the measurement is not easy. For example, a probe with a fine and complicated configuration is needed to implement the method as described above. Further, the probe itself may affect a distribution of the viscoelasticity. In contrast, the blood characteristics evaluation apparatus 1 according to the first embodiment is able to evaluate the characteristics of the blood sample with high accuracy based on the measurement result of the foaming state, and simply and easily perform evaluation without a need of a special probe or the like.

Furthermore, according to the first embodiment, the calculation function 12c calculates the index indicating the coagulation tendency of the blood sample, and evaluates the characteristics of the blood sample. Moreover, the output function 12d outputs the index that is calculated by the calculation function 12c. The user who is provided with the index as described above is able to perform determination on use of the blood coagulation related factor, such as heparin, in making a treatment plan or developing a procedure. For example, in a procedure using an artificial heart-lung machine, Drug-Eluting Stent placement, or the like, the user is able to determine necessity, a type, an amount of use, or the like of the blood coagulation related factor based on the evaluation result that is obtained by the blood characteristics evaluation apparatus 1.

Furthermore, as illustrated in FIG. 2, the control function 12a causes the blood sample to flow in the first flow path and the second flow path and causes the blood coagulation related factor to flow in the third flow path. Moreover, the control function 12a causes the foaming phenomena to occur in the blood sample that flows in the first flow path and the composite sample that flows in the fourth flow path in which the second flow path and the third flow path are merged. Therefore, according to the blood characteristics evaluation apparatus 1 of the first embodiment, it is possible to perform measurement of the foaming state in the blood sample and measurement of the foaming state in the composite sample in a parallel manner. In other words, according to the blood characteristics evaluation apparatus 1 of the first embodiment, it is possible to evaluate the characteristics of the blood sample in a reduced time.

Meanwhile, the control function 12a may control the flow rate in accordance with a timing at which the measurement function 12b measures the foaming state. For example, the control function 12a applies pulse voltage to the thermoelectric conversion element to cause the foaming phenomenon to occur, and reduce or stop the flow rate in the flow path after a lapse of a predetermined delay time. As one example, the control function 12a controls the flow in the flow path such that the flow rate becomes equal to or lower than a predetermined flow rate. Further, the measurement function 12b measures the foaming state at approximately the same timing at which the control function 12a reduces or stops the flow rate. In this manner, by reducing or stopping the flow rate at the time of measurement, it is possible to prevent the error in the bubble size or the like and evaluate the characteristics of the blood sample with high accuracy.

Furthermore, FIG. 2 illustrates the single flow path F11 in which the blood sample flows and the single flow path F14 in which the blood coagulation related factor flows, but the blood characteristics evaluation apparatus 1 may include a plurality of flow paths F11 and F14. Moreover, the plurality of flow paths may be merged in an arbitrary manner. Furthermore, the blood characteristics evaluation apparatus 1 is able to concurrently evaluate the blood characteristics under different conditions. For example, the blood characteristics evaluation apparatus 1 causes a blood coagulation related factor at a different concentration or a different kind of blood coagulation related factor to flow in each of the flow paths F14, and merges the flow path F11 in which the blood sample flows with each of the flow paths F14. Moreover, the blood characteristics evaluation apparatus 1 is able to concurrently perform measurement on the downstream sides of the respective flow paths F14 under different conditions.

### Second Embodiment

In the first embodiment as described above, the example illustrated in FIG. 2 is described as one example of the flow path in which the sample flows. In a second embodiment, a modification of the flow path will be described. A blood characteristics evaluation apparatus 1 according to the second embodiment has the same configuration as the blood characteristics evaluation apparatus 1 illustrated in FIG. 1, but is different from the blood characteristics evaluation apparatus 1 illustrated in FIG. 1 in that it includes a sample holder 16b illustrated in FIG. 6 instead of the sample holder 16a. FIG. 6 is a diagram illustrating a configuration example of flow paths according to the second embodiment. In the following, the same components as those described in the first embodiment will be denoted by the same reference symbols, and explanation thereof will be omitted.

The sample holder 16b may be manufactured with the same material and the same method as the sample holder 16a. For example, the control function 12a causes a blood sample to flow in a flow path F21 illustrated in FIG. 6 and causes a foaming phenomenon to occur in a part of the blood sample that flows in the flow path F21. For example, the sample holder 16b includes, as a heating part, a heater H21 at a position corresponding to a region R21 in FIG. 6. Further, the control function 12a applies thermal energy to the blood sample from the heater H21 and causes the foaming phenomenon to occur in the blood sample in the region R21 in the flow path F21. For example, the control function 12a applies thermal energy to the position corresponding to the region R21 in FIG. 2, and causes the foaming phenomenon to occur. The flow path F21 is one example of the first flow path.

Furthermore, the control function 12a causes a blood coagulation related factor to flow in a flow path F22. In the flowing, heparin will be described as one example of the blood coagulation related factor. For example, the control function 12a causes, s the blood coagulation related factor, heparin solution containing heparin at a predetermined concentration to flow in the flow path F22. The flow path F22 is one example of the second flow path.

As illustrated in FIG. 6, the flow path F21 and the flow path F22 are merged into a flow path F23. Specifically, in the flow path F23, a composite sample flows in which the blood sample that flows in the flow path F21 and the heparin solution that flows in the flow path F22 are mixed. The flow path F23 is one example of the third flow path. Similarly to the case of the region R21, the control function 12a causes the foaming phenomenon to occur in the composite sample in the region R22. For example, the sample holder 16a includes, as a heating part, a heater H22 FIG. 6 at a position corresponding to the region R22. Further, the control function 12a applies thermal energy to the composite sample from the heater H22, and causes the foaming phenomenon to occur in the composite sample that flows in the region R22 in the flow path F23.

The measurement function 12b measures the foaming state in the blood sample and the foaming state in the composite sample. For example, the measurement function 12b captures an image of the region R21 in which the foaming phenomenon has occurred, and measures a bubble size in the blood sample based on the image. Further, the measurement function 12b captures an image of the region R22 in which the foaming phenomenon has occurred, and measures a bubble size in the composite sample based on the image.

Meanwhile, a bubble that has generated in the region R21 disappears in a short time. In particular, if the foaming phenomenon has occurred due to film boiling, a time needed from foaming to defoaming is reduced. However, it is preferable to arrange a predetermined interval between the region R21 and the region R22 to prevent a bubble that has generated in the region R21 from remaining in the region R22 and being measured in the region R22.

Here, the processing circuitry 12 is able to repeatedly measure the foaming state in the composite sample in the region R22 while changing the blood coagulation related factor. For example, after causing a solution containing heparin at a predetermined concentration to flow in the flow path F22 and measuring the foaming state, the processing circuitry 12 causes a solution containing heparin at a different concentration or a solution containing a different kind of anticoagulant factor to flow in the flow path F22, and measures the foaming state again.

Alternatively, the processing circuitry 12 may perform measurement at a position on the downstream side of the region R22 by using a changed blood coagulation related factor. For example, the control function 12a causes a heparin solution L21 containing heparin to flow in the flow path F22. A concentration or a flow volume of the heparin in the heparin solution L2 is adjusted such that the concentration of the heparin in the composite sample as a mixture of the blood sample and the heparin solution L21 reaches the concentration C1. In this case, the measurement function 12b is able to measure the foaming state in the composite sample at the concentration C1 in the region R22.

Furthermore, the control function 12a causes a heparin solution L22 containing heparin to flow in a flow path F24 (not illustrated). Here, the flow path F24 is configured so as to be merged with the flow path F23. Moreover, a concentration or a flow volume of the heparin in the heparin solution L22 is adjusted such that the concentration of the heparin in a composite sample as a mixture of the composite sample that flows in the flow path F23 and the heparin solution L22 reaches the concentration C2. In this case, the measurement function 12b is able to measure the foaming state in the composite sample at the concentration C2 in a flow path F25 (not illustrated) in which the flow path F23 and the flow path F24 are merged. Meanwhile, the flow path F24 is one example of the second flow path. Further, the flow path F25 is one example of the third flow path.

Similarly, the control function 12a causes a heparin solution L23 containing heparin to flow in a flow path F26 (not illustrated). Here, the flow path F26 is configured so as to be merged with the flow path F25. Further, a concentration or a flow volume of the heparin in the heparin solution L23 is adjusted such that the concentration of the heparin in a composite sample as a mixture of the composite sample that flows in the flow path F25 and the heparin solution L23 reaches the concentration C3. In this case, the measurement function 12b is able to measure the foaming state in the composite sample at the concentration C3 in a flow path F27 (not illustrated) in which the flow path F25 and the flow path F26 are merged. Meanwhile, the flow path F26 is one example of the second flow path. Further, the flow path F27 is one example of the third flow path.

Specifically, the control function 12a causes the heparin solution to flow in each of the second flow paths, and causes the foaming phenomenon to occur in each of the composites samples that flows in the plurality of third flow paths in which the first flow path and the one or more second flow paths are merged. Further, the measurement function 12b measures the foaming state in the composite sample in each of the third flow paths, such as the flow path F23, the flow path F25, and the flow path F27. Here, the measurement function 12b is able to measure the foaming state in the blood sample in the flow path F21, the foaming state in the composite sample in the flow path F23, the foaming state in the composite sample in the flow path F25, and the foaming state in the composite sample in the flow path F27 at approximately the same time. In other words, the blood characteristics evaluation apparatus 1 is able to concurrently evaluate the blood characteristics under different conditions.

As described above, according to the second embodiment, the control function 12a causes the blood sample to flow in the first flow path and causes the blood coagulation related factor to flow in the second flow path. Further, the control function 12a causes the foaming phenomena to occur in the blood sample that flows in the first flow path and the composite sample that flows in the third flow path in which the first flow path and the second flow path are merged. Furthermore, the measurement function 12b measures the foaming state in the blood sample and the foaming state in the composite sample.

Moreover, the calculation function 12c evaluates the characteristics of the blood sample based on the measurement results of the foaming state in the blood sample and the foaming state in the composite sample. In other words, the blood characteristics evaluation apparatus 1 according to the second embodiment is able to provide a new method for evaluating the blood characteristics. Furthermore, the blood characteristics evaluation apparatus 1 according to the second embodiment is able to simply and easily evaluate the blood characteristics with high accuracy, similarly to the first embodiment. Moreover, as illustrated in FIG. 6, the sample holder 16b includes the smaller number of flow paths than the sample holder 16a. In other words, the blood characteristics evaluation apparatus 1 according to the second embodiment is able to more simply and easily evaluate the blood characteristics.

### Third Embodiment

While the first and the second embodiments have been described above, various different modes other than the embodiments as described above may be made.

For example, in the embodiments as described above, the case has been described in which the bubble size is measured based on the image of the region in which the foaming phenomenon has occurred. However, the embodiments are not limited to this example.

As one example, the measurement function 12b may measure the bubble size based on an amount of transmitted light in the region in which the foaming phenomenon has occurred. For example, the measurement function 12b causes the light source 13 to emit light toward the region in which the foaming phenomenon has occurred, and measures the amount of transmitted light by using the camera 14, a light receiving element (not illustrated), or the like. Here, light absorptivity in a bubble is lower than the blood sample and the composite sample, and therefore, if a large bubble is present in a light path, the amount of transmitted light increases. Therefore, the measurement function 12b is able to measure the bubble size in accordance with the amount of transmitted light.

As another example, the measurement function 12b may measure the bubble size based on an amount of reflected light from the region in which the foaming phenomenon has occurred. For example, the measurement function 12b causes the light source 13 to apply light toward the region in which the foaming phenomenon has occurred, and measures the amount of reflected light by using the camera 14, the light receiving element (not illustrated), or the like. Here, the reflected light from the region in which the foaming phenomenon has occurred is reflected light from an interface between the blood sample or the composite sample and the bubble. In other words, if a large bubble is present in a light path, the interface increases, so that the amount of reflected light increases. Therefore, the measurement function 12b is able to measure the bubble size in accordance with the amount of reflected light.

Alternatively, the measurement function 12b may measure the bubble size based on a temporal change (temporal profile) of the amount of transmitted light or the amount of reflected light. For example, the amount of transmitted light starts to increase after foaming is started, reaches a peak, gradually decreases, and returns to an original state that is observed before foaming. In other words, in the temporal change of the amount of transmitted light, a time needed from start of an increase in the amount of transmitted light to return to an original value corresponds to a duration from foaming to defoaming. Furthermore, in general, a time needed from foaming to defoaming increases with an increase in the bubble size. Thus, the measurement function 12b is able to measure the bubble size by acquiring the temporal change of the amount of transmitted light and measuring the time from start of an increase in the amount of transmitted light to return to the original value, a time needed until peak, or the like. Similarly, the measurement function 12b is able to measure the bubble size based on the temporal change of the amount of reflected light.

Meanwhile, the temporal change of the amount of transmitted light or the amount of reflected light may be acquired by measuring light intensity with respect to single foaming or measuring light intensity with respect to multiple forming. In the former method, for example, a measurement function 112b continuously or repeatedly measures light intensity while causing the light source 13 to continuously or repeatedly emit light with respect to single foaming, and measures a temporal change of the light intensity. In contrast, in the latter method, for example, the measurement function 112b acquires the temporal change of the light intensity by performing a set of measurement, in which light intensity is measured once or a plurality of number of times with respect to single foaming, a plurality of number of times while changing a delay time between foaming and light intensity measurement. In this case, the measurement function 112b is able to synchronize a light emission timing of the light source 13 and a light intensity measurement timing by causing the light source 13 to emit light in accordance with the delay time between the foaming and the light intensity measurement.

Furthermore, in the embodiments as described above, the case has been described in which the bubble size is measured as the foaming state. However, the embodiments are not limited to this example. For example, the measurement function 12b may measure a shape of a bubble as the foaming state. Here, the shape of the bubble and the characteristics of the blood sample have a correlation, and therefore, the calculation function 12c is able to evaluate the characteristics of the blood sample based on the measurement result of the shape of the bubble. For example, deformation of the bubble is more prevented with an increase in viscosity, so that the bubble is likely to be formed in a spherical shape. Therefore, the calculation function 12c is able to calculate viscosity of the blood sample based on the measurement result the bubble shape.

As another example, the measurement function 12b may measure, as the foaming state, ejection characteristics due to foaming. Specifically, the control function 12a heats the blood sample in a hollow member, such as a nozzle. Accordingly, film boiling occurs in a part of the blood sample, pressure is generated in the member, and the blood sample is ejected. Here, the measurement function 12b measures, as the foaming state, the ejection characteristics, such as the amount or velocity of the ejected blood sample. The same applies to the composite sample, so that it is possible to measure the ejection characteristics due to foaming.

Furthermore, in the embodiments as described above, the case has been described in which the blood sample is stirred before measurement. Here, the blood characteristics evaluation apparatus 1 may measure the foaming state without performing stirring and determine necessity of stirring of the blood sample in accordance with a measurement result.

Specifically, first, the control function 12a causes the foaming phenomenon to occur in the blood sample in a non-stirred state. Subsequently, the measurement function 12b measures the foaming state in the blood sample. Then, the calculation function 12c calculates variation (dispersion) of the measurement result of the foaming state. Here, if the variation is smaller than a threshold, the control function 12a determines that the blood sample needs to be stirred. In other words, if the variation is smaller than the threshold, the calculation function 12c evaluates the characteristics of the blood sample based on the measurement result of the foaming state in the blood sample in the non-stirred state. Through the process as described above, a stirring step is omitted if the stirring is not needed, so that it is possible to evaluate the characteristics of the blood sample in a reduced time.

In contrast, if the variation is larger than the threshold, the control function 12a determines that the blood sample needs to be stirred. In other words, if the variation is smaller than the threshold, the control function 12a stirs the blood sample, and causes the foaming phenomenon to occur in the stirred blood sample. Furthermore, the measurement function 12b measures the foaming state in the blood sample. Then, the calculation function 12c evaluates the characteristics of the blood sample based on the measurement result of the foaming state in the stirred blood sample. Through the process as described above, it is possible to ensure that the measurement condition is unified, and further improve the evaluation accuracy.

Moreover, in the embodiments as described above, the case has been described in which the characteristics of the blood sample is evaluated by calculating the index indicating the coagulation tendency of the blood sample. However, the embodiments are not limited to this example. For example, the calculation function 12c may evaluate the characteristics of the blood sample by calculating an index other than the index indicating the coagulation tendency of the blood sample. As one example, for example, the calculation function 12c may calculate, as the index other than the index indicating the coagulation tendency of the blood sample, viscosity, viscoelasticity, surface tension, composition, or the like of the blood sample.

For example, it is known that the size of the bubble that is generated in liquid is reduced with an increase in viscosity of the liquid. Therefore, the calculation function 12c may calculate, as an evaluation result of the characteristics of the blood sample, viscosity based on the bubble size.

For example, the calculation function 12c generates association information in which the viscosity and the bubble size are associated with each other. As one example, the control function 12a causes an arbitrary liquid sample for which viscosity is already known to flow in the flow paths illustrated in FIG. 2 and FIG. 6. Further, the measurement function 12b causes the foaming phenomenon to occur in the liquid sample. Furthermore, the calculation function 12c generates association information by associating the size of a bubble in the liquid sample with certain viscosity that is already known, and stores the association information in the memory 11. Then, if the foaming state in the blood sample is measured, the calculation function 12c is able to calculate the viscosity of the blood sample based on the association information that is read from the memory 11.

For example, in the embodiments as described above, the slope of the regression equation between the concentration of the heparin and the bubble size has been described as an example of the index indicating the coagulation tendency of the blood sample. However, the embodiments are not limited to this example. For example, the calculation function 12c may calculate a statistical value, such as a variance value, with respect to the bubble size when the concentration of the heparin is changed, and adopt the statistical value as the index indicating the coagulation tendency of the blood sample. Furthermore, there is a correlation between the coagulation tendency and the size of the bubble, so that the calculation function 12c may calculate a value corresponding to the size of the bubble as the index indicating the coagulation tendency of the blood sample.

Furthermore, in the embodiments as described above, the case has been described in which both of measurement of the foaming state in the blood sample and measurement of the foaming state in the composite sample in which the blood sample and the blood coagulation related factor are mixed are performed, but one of the measurement may be omitted.

For example, the control function 12a omits a process of causing the foaming phenomenon to occur in the composite sample, and causes the foaming phenomenon to occur in only the blood sample. Furthermore, the measurement function 12b measures the foaming state in the blood sample. Moreover, the calculation function 12c evaluates the characteristics of the blood sample based on the measurement result of the foaming state in the blood sample. For example, the calculation function 12c is able to calculate an index, such as the viscosity, in accordance with the size of the bubble, or the index indicating the coagulation tendency of the blood sample. The calculation function 12c may calculate the index by referring to a lookup table corresponding to the number of blood cells.

Moreover, for example, the control function 12a omits a process of causing the foaming phenomenon to occur in the blood sample, and causes the foaming phenomenon to occur in only the composite sample. Furthermore, the measurement function 12b measures the foaming state in the composite sample. Moreover, the calculation function 12c evaluates the characteristics of the blood sample based on the measurement result of the foaming state in the composite sample. For example, the calculation function 12c is able to calculate the index, such as the viscosity, in accordance with the size of the bubble or the index indicating the coagulation tendency of the blood sample. For example, if the foaming state is measured for a plurality of composites samples for each of which the concentration of the heparin is changed, the calculation function 12c is able to calculate the slope of the regression equation between the concentration of the heparin and the bubble size.

The term “processor” used in the description above indicates, for example, circuitry, such as a CPU, a Graphics Processing Unit (GPU), an Application Specific Integrated Circuit (ASIC), or a programmable logic device (for example, a Simple Programmable Logic Device (SPLD), a Complex Programmable Logic Device (CPLD), or a Field Programmable Gate Array (FPGA)). If the processor is, for example, a CPU, the processor implements the functions by reading a program stored in memory circuitry and executing the program. In contrast, if the processor is, for example, an ASIC, the functions are directly incorporated, as logical circuitry, in circuitry of the processor, rather than storing the program in the memory circuitry. Meanwhile, each of the processors of the embodiments need not always be configured as single circuitry for each processor, but may be configured as a single processor by combining a plurality of independent circuitry to implement the functions. Further, a plurality of components in each of the drawings may be integrated into a single processor to implement the functions.

Furthermore, in FIG. 1, it has been explained that the single memory 11 stores therein the program corresponding to each of the processing functions of the processing circuitry 12. However, the embodiments are not limited to this example. For example, it may be possible to arrange the plurality of memories 11 in a distributed manner and cause the processing circuitry 12 to read a corresponding program from each of the memories 11. Furthermore, it may be possible to directly incorporate the program in circuitry of the processor, rather than storing the program in the memory 11. In this case, the processor implements the functions by reading the program incorporated in the circuitry and executing the program.

The components of the apparatuses illustrated in the drawings are conceptual function, and need not be physically configured in the manner illustrated in the drawings. In other words, specific forms of distribution and integration of the apparatuses are not limited to those illustrated in the drawings, and all or part of the apparatuses may be functionally or physically distributed or integrated in arbitrary units depending on various loads or use conditions. Further, all or an arbitrary part of the processing functions implemented by the apparatuses may be realized by a CPU and a program analyzed and executed by the CPU, or may be realized by hardware using wired logic.

Furthermore, the blood characteristics evaluation method described in the above embodiments may be implemented by causing a computer, such as a personal computer or a workstation, to execute a program that is prepared in advance. The program may be distributed via a network, such as the Internet. Moreover, the program may be recorded in a non-transitory computer readable recording medium, such as a hard disk, a flexible disk (FD), a compact disc-read only memory (CD-ROM), a magneto optical disk (MO), or a digital versatile disk (DVD), and executed by being read from the recording medium by a computer.

According to at least one of the embodiments as described above, it is possible to provide a new method for evaluating blood characteristics.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

In relation to the embodiments as described above, following notes are disclosed as one aspect and selective features of the disclosed technology.

Note. 1

A blood characteristics evaluation apparatus includes processing circuitry configured to cause a foaming phenomenon to occur in a blood sample, measure a foaming state in the blood sample, and calculate an index indicating a coagulation tendency of the blood sample based on a measurement result of the foaming state in the blood sample.

Note. 2

The processing circuitry may cause a foaming phenomenon to occur in the blood sample and a composite sample in which the blood sample and a blood coagulation related factor are mixed, measure a foaming state in the blood sample and a foaming state in the composite sample, and calculate the index based on measurement results of the foaming state in the blood sample and the foaming state in the composite sample.

Note. 3

The processing circuitry may cause the blood sample to flow in a first flow path and a second flow path, cause the blood coagulation related factor to flow in a third flow path, and cause the foaming phenomenon to occur in each of the blood sample that flows in the first flow path and the composite sample that flows in a fourth path in which the second flow path and the third flow path are merged.

Note. 4

The processing circuitry may approximately simultaneously measure the foaming state in the blood sample that flows in the first flow path and the foaming state in the composite sample that flows in the fourth flow path.

Note. 5

The processing circuitry may cause the blood sample to flow in a first flow path, cause the blood coagulation related factor to flow in a second flow path, and generate the foaming phenomenon to occur in each of the blood sample that flows in the first flow path and the composite sample that flows in a third flow path in which the first flow path and the second flow path are merged.

Note. 6

The processing circuitry may cause the blood coagulation related factor to flow in each of a plurality of second flow paths, and cause the foaming phenomenon to occur in the plurality of composite samples that flows in the third flow path in which the first flow path and the one or more second flow paths are merged.

Note. 7

The blood coagulation related factor may be heparin.

Note. 8

The processing circuitry may cause the foaming phenomenon to occur by applying thermal energy.

Note. 9

The c processing circuitry may calculate the index based on the measurement result and the number of blood cells in the blood sample.

Note. 10

The processing circuitry may cause the foaming phenomenon to occur in the blood sample in which the number of blood cells is adjusted to a predetermined value.

Note. 11

The processing circuitry may cause the foaming phenomenon to occur in the blood sample that has been stirred.

Note. 12

The processing circuitry may cause the foaming phenomenon to occur in the blood sample that has not been stirred, measure the foaming state in the blood sample, and calculate variation in a measurement result of the foaming state, wherein
if the variation is smaller than a threshold, the processing circuitry may calculate the index based on a measurement result of the foaming state in the blood sample that is not stirred,
and if the variation is larger than the threshold, the processing circuitry may stir the blood sample, and cause the foaming phenomenon to occur in the stirred blood sample.

Note. 13

The processing circuitry may measure the foaming state in the blood sample and eliminate an outlier from a measurement result of the foaming state, and calculate the index based on the measurement result from which the outlier is eliminated.

Note. 14

The processing circuitry may display an error range of the index together with the index.

Note. 15

The processing circuitry may measure a bubble size as the foaming state.

Note. 16

The processing circuitry may measure the bubble size based on one of an image of a region in which the foaming phenomenon has occurred, an amount of transmitted light in the region, and an amount of reflected light from the region.

Note. 17

The processing circuitry may measure the bubble size based on a temporal change of one of the amount of transmitted light and the amount of reflected light.

Note. 18

The processing circuitry may measure, as the foaming state, ejection characteristics due to foaming.

Note. 19

A blood characteristics evaluation apparatus includes processing circuitry configured to cause a foaming phenomenon to occur in a composite sample in which a blood sample and a blood coagulation related factor are mixed, measure a foaming state in the composite sample, and evaluate characteristics of the blood sample based on a measurement result of the foaming state in the composite sample.

Note. 20

The processing circuitry may calculate, as the evaluation, an index indicating a coagulation tendency of the blood sample.

Note. 21

The processing circuitry may cause a foaming phenomenon to occur in the blood sample and the composite sample, measure the foaming state in the blood sample and the foaming state in the composite sample, and calculate the evaluation based on measurement results of the foaming state in the blood sample and the foaming state in the composite sample.

Note. 22

A blood characteristics evaluation method includes:
causing a foaming phenomenon to occur in a blood sample;
measuring a foaming state in the blood sample; and
calculating an index indicating a coagulation tendency of the blood sample based on a measurement result of the foaming state in the blood sample.

Note. 23

A blood characteristics evaluation method includes:
causing a foaming phenomenon to occur in a composite sample in which a blood sample and a blood coagulation related factor are mixed;
measuring a foaming state in the composite sample; and
evaluating characteristics of the blood sample based on a measurement result of the foaming state in the composite sample.

Note. 24

A program that causes a computer to implement each of the components of the blood characteristics evaluation apparatus as described above.

What is claimed is:

1. A blood characteristics evaluation apparatus comprising:
processing circuitry configured to:
cause a foaming phenomenon to occur in a blood sample,
measure a foaming state in the blood sample, and
calculate an index indicating a coagulation tendency of the blood sample based on a measurement result of the foaming state in the blood sample.

2. The blood characteristics evaluation apparatus according to claim 1, wherein the processing circuitry
causes a foaming phenomenon to occur in the blood sample and a composite sample in which the blood sample and a blood coagulation related factor are mixed,
measures a foaming state in the blood sample and a foaming state in the composite sample, and
calculates the index based on measurement results of the foaming state in the blood sample and the foaming state in the composite sample.

3. The blood characteristics evaluation apparatus according to claim 2, wherein the processing circuitry
causes the blood sample to flow in a first flow path and a second flow path,
causes the blood coagulation related factor to flow in a third flow path, and
causes the foaming phenomenon to occur in each of the blood sample that flows in the first flow path and the composite sample that flows in a fourth path in which the second flow path and the third flow path are merged.

4. The blood characteristics evaluation apparatus according to claim 3, the processing circuitry approximately simultaneously measures the foaming state in the blood sample that flows in the first flow path and the foaming state in the composite sample that flows in the fourth flow path.

5. The blood characteristics evaluation apparatus according to claim 2, wherein the processing circuitry
causes the blood sample to flow in a first flow path,
causes the blood coagulation related factor to flow in a second flow path, and
generates the foaming phenomenon to occur in each of the blood sample that flows in the first flow path and the composite sample that flows in a third flow path in which the first flow path and the second flow path are merged.

6. The blood characteristics evaluation apparatus according to claim 5, wherein the processing circuitry causes the blood coagulation related factor to flow in each of the plurality of second flow paths, and causes the foaming phenomenon to occur in the plurality of composite samples that flows in the third flow path in which the first flow path and the one or more second flow paths are merged.

7. The blood characteristics evaluation apparatus according to claim 2, wherein the blood coagulation related factor is heparin.

8. The blood characteristics evaluation apparatus according to claim 1, wherein the processing circuitry causes the foaming phenomenon to occur by applying thermal energy.

9. The blood characteristics evaluation apparatus according to claim 1, wherein the processing circuitry calculates the index based on the measurement result and number of blood cells in the blood sample.

10. The blood characteristics evaluation apparatus according to claim 1, wherein the processing circuitry causes the foaming phenomenon to occur in the blood sample in which the number of blood cells is adjusted to a predetermined value.

11. The blood characteristics evaluation apparatus according to claim 1, wherein the processing circuitry causes the foaming phenomenon to occur in the blood sample that has been stirred.

12. The blood characteristics evaluation apparatus according to claim 1,
the processing circuitry measures the foaming state in the blood sample, and eliminates an outlier from a measurement result of the foaming state, and
the processing circuitry calculates the index based on the measurement result from which the outlier is eliminated.

13. The blood characteristics evaluation apparatus according to claim 1, wherein the processing circuitry displays an error range of the index together with the index.

14. the blood characteristics evaluation apparatus according to claim 1, wherein the processing circuitry measures a bubble size as the foaming state.

15. The blood characteristics evaluation apparatus according to claim 14, wherein the processing circuitry measures the bubble size based on one of an image of a region in which the foaming phenomenon has occurred, an amount of transmitted light in the region, and an amount of reflected light from the region.

16. The blood characteristics evaluation apparatus according to claim 15, wherein the processing circuitry measures the bubble size based on a temporal change of one of the amount of transmitted light and the amount of reflected light.

17. The blood characteristics evaluation apparatus according to claim 1, wherein the processing circuitry measures, as the foaming stats ejection characteristics due to foaming.

18. A blood characteristics evaluation apparatus comprising:
processing circuitry configured to:
cause a foaming phenomenon to occur in a composite sample in which a blood sample and a blood coagulation related factor are mixed;
measure a foaming state in the composite sample; and
evaluate characteristics of the blood sample based on a measurement result of the foaming state in the composite sample.

19. The blood characteristics evaluation apparatus according to claim 18, wherein the processing circuitry calculates, as the evaluation, an index indicating a coagulation tendency of the blood sample.

20. The blood characteristics evaluation apparatus according to claim 18, wherein the processing circuitry
causes a foaming phenomenon to occur in the blood sample and the composite sample,
measures the foaming state in the blood sample and the foaming state in the composite sample, and
calculates the evaluation based on measurement results of the foaming state in the blood sample and the foaming state in the composite sample.

21. A blood characteristics evaluation method comprising:
causing a foaming phenomenon to occur in a blood sample;
measuring a foaming state in the blood sample; and
calculating an index indicating a coagulation tendency of the blood sample based on a measurement result of the foaming state in the blood sample.

22. A blood characteristics evaluation method comprising:
causing a foaming phenomenon to occur in a composite sample in which a blood sample and a blood coagulation related factor are mixed;
measuring a foaming state in the composite sample; and
evaluating characteristics of the blood sample based on a measurement result of the foaming state in the composite sample.

23. A non-transitory computer readable recording medium having stored therein a program that causes a computer to perform a process comprising:
causing a foaming phenomenon to occur in a blood sample;
measuring a foaming state in the blood sample; and
calculating an index indicating a coagulation tendency of the blood sample based on a measurement result of the foaming state in the blood sample.

24. A non-transitory computer readable recording medium having stored therein a program that causes a computer to perform a process comprising:
causing a foaming phenomenon to occur in a composite sample in which a blood sample and a blood coagulation related factor are mixed;
measuring a foaming state in the composite sample; and
evaluating characteristics of the blood sample based on a measurement result of the foaming state in the composite sample.
